(19) United States
(12) Patent Application Publication
(10) Pub. No.: US 2026/0355415 A1
(43) Pub. Date: Nov. 12, 2026

(54) TROCHOIDAL MEDICAMENT PUMP DEVICE: MATERIAL AND GEOMETRY

(71) Applicant: MEDICO INVEST AG, St. Gallen (CH)

(72) Inventors: Herbert BECHTOLD, Denkingen (DE); Stefan GAUL, Stetten (CH); Tom OAKLEY, Cambridgeshire (GB); Thomas WYATT, Cottenham (GB); Kamaal DE SILVA, Cambridge (GB)

(21) Appl. No.: 19/469,854

(22) PCT Filed: Mar. 27, 2024
(86) PCT No.: PCT/EP2024/058308

§ 371 Date: Sep. 26, 2025

(30) Foreign Application Priority Data
Mar. 31, 2023 (EP) 23166023.4

(51) Int. Cl. A61M 5/145; A61M 5/14

(52) U.S. Cl. CPC A61M 5/145; A61M 5/1413; A61M 2205/0216; A61M 2205/103

(57) ABSTRACT

Trochoidal medicament pump device, and a giving set having included said trochoidal medicament pump device, which has a particular geometry and material selection. The trochoidal medicament pump device and the giving set having included said trochoidal medicament pump device provides a trochoidal medicament pump device and giving set having included said trochoidal medicament pump device with improved delivery properties, exactness, reliability, and lifetime.

### FIELD OF THE INVENTION

The present invention relates to a trochoidal medicament pump device, which has a particular geometry and material selection, and further relates to a giving set having included said trochoidal medicament pump device. The trochoidal medicament pump device and the giving set having included said trochoidal medicament pump device according to the invention provide a trochoidal medicament pump device and giving set having included said trochoidal medicament pump device with improved delivery properties, accuracy, reliability, and lifetime.

### BACKGROUND OF THE INVENTION

Trochoidal pump devices are known for pumping applications, e.g., from U.S. Pat. No. 4,137,024 which describes a rotary piston mechanism such as compressors and expansion engines, and from JP-S-62-17322, which describes a rotary piston type compressor. Trochoidal pump devices are also known for pumping applications in refrigerators, as described e.g., in U.S. Pat. No. 6,520,754, which describes a compressor for a refrigerator for household applications. U.S. Pat. No. 6,168,405 B1 describes a vacuum cleaner with a Wankel-type pump for pumping air wherein the chamber has an epitrochoidal planform satisfying a particular equation.

A trochoidal pump device for medical applications is also known, e.g., from WO 2019/057570, which describes a rotary pump for use in a medicament delivery device.

Further, WO 2021/109759 A1 describes a triangular rotor pump, which however does not have any relation to medicament delivery. Further, WO 2019/057570 A2 describes rotary pump and a medical device for the administration of one or more liquid medicaments to a patient, which however does not have any modularity and exchangeability of the pump and its drive. U.S. Pat. No. 9,511,186 B1 describes an oval rotor medicament injection system with a pump, which however does not have a trochoidal shape. US 2021/353854 A1 describes an infusion pump with a pump engine, which however does not have a receptacle for a trochoidal pump.

US 2018/010612 A1 describes a flexible impeller pump, which however does not have a trochoidal pump. US 2019/143039 A1 describes an infusion device with different spiral or bent needle shapes. WO 01/39816 A2 describes an infusion pump, which however does not have a trochoidal pump.

Over the known prior art is can be considered as an object to provide a trochoidal medicament delivery device with improved delivery properties, higher accuracy, increased reliability, and longer lifetime.

### SUMMARY OF THE INVENTION

The present invention provides a trochoidal medicament pump device and a giving set including said trochoidal medicament delivery device, according to the independent claims, whereas further embodiments are incorporated in the dependent claims.

According to an embodiment there is provided a trochoidal medicament pump device comprising a pump body with an in-port for entering a medicament to be administered and an out-port for administering a medicament, a pump rotor having a triangular convex shape and having an eccentric trajectory upon rotation, i.e. revolving around an axis, within the pump body, wherein the pump rotor has three equidistant apexes, each two thereof are connected with a convex curved line, wherein the pump body has a pump chamber, wherein the pump chamber has a first lobe forming a first inner wall portion of a pump volume of the pump chamber with a first inlet in fluid communication with the in-port and a first outlet in fluid communication with the out-port with the pump rotor eccentrically rotating, i.e. revolving around an axis, so that the rotor with the first wall portion forms upon rotation a first suction volume in fluid communication to the first inlet and a first expelling volume in fluid communication to the first outlet, wherein the pump rotor has a rigid rotor core and an elastic or elastomeric over-mold over at least sealing lines along the apexes and sealing lines along the convex curved lines, wherein the over-mold is made of an elastic or elastomeric material of a first material class, wherein the first inner wall portion is made of a material of a second material class, wherein the outer dimensions of the pump rotor with the over-mold, including a top and bottom, are equivalent or larger than the corresponding inner dimensions of the pump chamber, wherein the apexes of the eccentrically rotating pump rotor trace a trochoidal line corresponding to a trochoidal shape that is equivalent or larger than the corresponding inner dimensions of the pump chamber.

Thus, the trochoidal medicament pump device may provide a reliable delivery of a medicament. Within the context of rotary pumps, a trochoid is defined as the plane curve that is generated by following the trajectory of a point fixed relative to one circle, where that circle rolls along another fixed circle. In the literature, this is also sometimes described as an epitrochoid or hypotrochoid, depending on whether the rolling circle contacts externally or internally with the fixed circle respectively. Thus, a trochoidal shape is one that has been generated using this geometrical method. The larger dimensions of the rotor in combination with the over-mold of an elastic or elastomeric material provide a reliable sealing over the pump chamber. It is the intention to make the pump chamber airtight, so that the pressure can get low enough to draw fluid up tubes and move, e.g., syringe/cartridge plungers, and to reduce any variability of dosage over time, even with any external influences. The rigid rotor core maintains the general geometry of the rotor and allows a reliable coupling of the rotor to the driving components, which drive the rotor over the pump body. Rigid means that the material is non-elastomeric, or at least less elastomeric than the elastomeric over-mold. The elastomeric over-mold provides a reliable sealing regardless in which rotational position the rotor is over the pump body. Upon eccentric rotation of the rotor, the rotor forms a suction chamber being in fluid communication with the inlet so that a fluid can enter the suction chamber. The eccentric rotation enlarges the suction volume upon further rotation so that a fluid is sucked into the suction volume. The suction volume rotates with the rotor until one of the apexes passes the inlet and thus closes the suction volume. As soon as the volume filled with fluid comes into communication with the outlet, the volume becomes an expelling volume and upon further rotation, the volume decreases and thus expels the fluid. At the same time, the following part of the triangular geometry of the rotor already operates as a suction volume and the previously described procedure repeats. Thus, the suction volume is the volume as long as it is connected to the inlet port. As soon as the volume is connected to the outlet, it becomes the expelling volume. The geometry of the trochoidal lobe in combination with the convex curved lines result in a very low volume after the expelling and before the next apex passes the outlet, so that almost all fluid or ideally all fluid is expelled. In some phases of the rotor rotation three portions of the rotor are in permanent contact with the inner wall of the first lobe, so that the rotor together with the inner wall always form a suction volume and an expelling volume at the same time, with the repeated procedure that the sucked fluid in the suction volume is transported until the volume becomes the expelling volume and the fluid is expelled, while the following geometry has just formed the following suction volume. The principle is analogue to the so called Wankel piston.

It should be noted that the terms apex and convex curved line is to be understood as seen from a top view, i.e., without the third dimension. In the third dimension, the apex is a line, and the convex curved line is a convex curved surface. This surface is limited by the two adjacent apex lines as well as both curved convex lines connecting the end points of both apex lines.

According to an embodiment, the over-mold is made of a biocompatible elastomeric material of the first material class, and the first inner wall portion is made of a biocompatible material of the second material class.

Thus, the pump and all its components which come into contact with a medicament liquid or fluid are resistant against deterioration or decomposition so that no substances from the rotor, in particular the over-mold, or the pump body, in particular the inner wall, enter the medicament liquid.

According to an embodiment the rigid rotor core has an undercut geometry, wherein the undercut geometry along each a convex curved line has an undercut orthogonal to the rotational axis of the rotor.

Thus, the core with the undercut geometry offers a maximized surface for e.g. chemical bonding like gluing as well as a maximized undercut with narrow openings so that the over-mold elastic material cannot slip back through the slot even under high pressure and high tensile forces applied to the over-mold material and/or under-pressure in the active pumping chamber that would allow elastic material to slip back in case bonding is not sufficient. Thus, the undercut serves as a further safety measure in addition to a surface bonding of the over-mold to the core. The occurring forces, irrespective of their origin, may be received in part by the surface bonding and in part by the undercut. If one of the surface bonding and the undercut fails, the remaining one may take over.

According to an embodiment the rigid rotor core along each a convex curved line has a plurality of recesses being adapted for providing access for an over-mold to the undercut geometry.

Thus, the adhesion of the over-mold at the core of the rotor can be improved, and detaching of the over-mold from the core can be avoided. This keeps the geometry in shape and allows a reliable feeding and delivery of a medicament liquid or fluid and may contribute to a more reliable sealing between chambers and may avoid potential air entering the active pumping chamber. Further, a better manufacturing of the over-mold is achieved and an improved bonding of over-mold above the undercut geometry and under the undercut geometry in radial direction. According to an embodiment smaller recesses and wider recesses may be provided providing a safe mechanical fixation of the elastic material, e.g., LSR. The dimension of the slots, i.e., a gap width, depends on the elasticity and shear force of the elastic over-mold material. Cylindrical holes parallel to the rotation axis may be provided for fixing top portions and bottom portions of the over-mold under the undercut geometry. The dimensions of these holes may depend on the flow index of the unset over-mold material and the shear forces.

According to an embodiment, the first material class is a group consisting of liquid silicone rubber LSR, ethylene propylene diene monomer rubber EPDM, thermoplastic elastomer TPE), thermoplastic polyurethane (TPU), liquid crystalline polymer (LCP), butyl rubbers and nitrile rubbers.

Thus, flexible materials with adequate sealing properties, low friction over a pump body material and durability may be provided. It revealed that liquid silicone rubber LSR is particularly suitable for 2 k molding especially in medical devices with the requirements of multiple sterilization processes not losing the physical properties. It revealed that ethylene propylene diene monomer rubber EPDM is particularly suitable for polar liquids. It revealed that TPE is particularly suitable for short time use, and has a larger compression set, has lower friction and a bonding compatibility with, e.g., a glass filled material. It revealed that TPU is particularly suitable for normal usage for tubes and bags and membranes. It revealed that butyl rubbers are suitable for cartridge plungers and nitrile rubbers for sealing elements.

According to an embodiment the first material class is a self-lubricating liquid silicone rubber LSR.

Thus, the friction can permanently be reduced upon rotation. According to an embodiment the self-lubricant LSR is biocompatible.

According to an embodiment the first material is a liquid silicone rubber LSR which is treated with a medical grade and bio compatible lubricant, in particular a medical grade and bio compatible silicone oil.

Thus, the friction can permanently be reduced upon rotation.

According to an embodiment, the over-mold is made of liquid silicone rubber LSR with a hardness between 10 and 100 Shore (ShA), in particular between 20 and 80 Shore (ShA), in particular between 40 and 60 Shore (ShA), in particular of 50+/−5 Shore (ShA).

Thus, a good balance may be achieved between reliable sealing an acceptable friction which is useful for operating the pump reliably with reasonable power effort. It revealed that using for the over-mold a LSR with a hardness between 50 and 100 Shore (ShA), and in particular between 60 and 70 Shore (ShA), this LSR configuration is suitable for fulfilling the requirements for low torque, minimal energy consumption, best fit in wear, friction, noise, sterilization, biocompatibility, lifetime usage and large temperature and humidity range usage.

According to an embodiment the rotor has an internal sealing lip which is provided on a at least one side of the rotor which is orthogonal to the rotational axis of the rotor, wherein the internal sealing lip is provided on the respective side radially between a gear section of the rotor and the convex curved lines.

Thus, additionally to a main sealing line along the convex curved lines, a further backup seal may be provided, which may be set back compared to the main seal so that it has a lower friction than the main seal. The internal seal lip may serve as a safety seal to prevent possible air entry through the eccentric position opening. This lip may be arranged cylindrically around the eccentric bearing, set back compared to the main seal with less pressure on the housing.

According to an embodiment, the second material class is a group consisting of polyethylene terephthalate PET, polycarbonate PC, Tritan, cycloolefin copolymer COC, PBT, PPS, and PEI.

Thus, rigid materials with adequate sealing properties over the rotor material, low friction over a pump rotor material and durability may be provided. Tritan is a copolymer made from three monomers: dimethyl terephthalate (DMT), cyclohexanedimethanol (CHDM), and 2,2,4,4-tetramethyl-1,3-cyclobutanediol (CBDO). It revealed that polyethylene terephthalate PET is particularly suitable for long-term use and durability. It revealed that polycarbonate PC is particularly suitable for short storage time. It revealed that Tritan is particularly suitable for all kind of drugs and liquids with a long storage lifetime without quality loss. It revealed that COC (cycloolefin copolymer) is particularly suitable for most of the liquids and drugs. It revealed that PBT is comparable to PET for being particularly suitable for long-term use and durability. It revealed that PPS is particularly suitable for extreme temperature ranges with high accuracy, and PEI is particularly suitable for its high temperature resistance. In general, these materials are also suitable for essential requirement such as sterilization, biocompatibility, lifetime usages and large temperature and humidity range usage.

According to an embodiment, the biocompatible material of the second material class is a Tritan compounded with a medical grade absorber.

Thus, it is possible to absorb light of a welding laser in order to convert the light radiation into heat and melt the material for welding. The absorbent may also be positioned at particular sections where a welding is intended to take place, whereas areas where welding should be avoided are not provided with an absorbent.

According to an embodiment, the absorbent is adapted for absorbing an optical radiation of a welding laser, in particular an optical radiation of a wavelength between 960 nm and 1000 nm, in particular a wavelength of 980 nm+/−10 nm.

Thus, the light absorption can be carried out at wavelengths of an infrared optical laser.

According to an embodiment the rotor core is made of a material of the second material class group filled with reinforcing fibers.

Thus, the core may have a higher stability.

According to an embodiment the rotor core is made of a material including PBT and 30+/−10 weight % glass fibers.

Thus, a strong core may be provided. It should be noted that instead of glass fibers also carbon fibers may be used.

According to an embodiment, the outer dimensions of the pump rotor with the over-mold are larger than the corresponding inner dimensions of contacted portions of the pump chamber by a factor, which factor is between 1.01 and 1.1, in particular between 1.01 and 1.05, in particular between 1.02 and 1.03.

Thus, a reliable sealing can be established with a reasonable friction, which allows operation with reasonable power demand. It revealed that a factor between 1.01 and 1.1, in particular between 1.01 and 1.05, in particular between 1.02 and 1.03 is suitable for liquid viscosities up to 50 cP (centipoise).

According to an embodiment, the factor in the radial direction is between 1.01 and 1.1, in particular between 1.01 and 1.05, in particular between 1.02 and 1.03.

Thus, a reliable sealing in the radial direction can be achieved. Radial direction is the direction orthogonal to the rotational axis, which corresponds to the direction orthogonal of the apex lines abutting the wall portions of the pump chamber. It revealed that a factor between 1.01 and 1.1, in particular between 1.01 and 1.05, in particular between 1.02 and 1.03 is suitable for drugs between viscosities from 1 cP to 50 cP and chamber pressures up to 10 bar (1 MPa).

According to an embodiment, the factor in the axial direction is between 1.01 and 1.1 in particular between 1.01 and 1.05, in particular between 1.01 and 1.03.

Thus, a reliable sealing in the axial direction can be achieved. Axial direction is the direction parallel to the rotational axis, which corresponds to the direction parallel to the apex lines abutting the wall portions of the pump chamber, which corresponds to the direction orthogonal of the bended lines abutting the inner wall of the cover and bottom of the pump chamber.

According to an embodiment, the outer dimensions of the pump rotor with the over-mold in radial direction are at least 0.01 mm larger than the corresponding inner dimensions of contacted portions of the pump chamber.

Thus, a reliable seal under applied pressure may be achieved in radial direction.

According to an embodiment, the outer dimensions of the pump rotor with the over-mold in axial direction are at least 0.07 mm larger than the corresponding inner dimensions of contacted portions of the pump chamber.

Thus, a reliable seal under applied pressure may be achieved in axial direction.

According to an embodiment the over-mold at each of the convex curved lines has a closed gas reservoir, wherein the gas reservoir is filled with gas under pressure higher than atmospheric pressure, in particular at a pressure between 1.2*10\hat{ }5 Pa and 2.0*10\hat{ }5 Pa, in particular at a pressure of 1.3*10\hat{ }5 Pa+/−0.2*10\hat{ }5 Pa.

Thus, a volume change of the pump chamber upon rotation can be compensated. As a liquid usually is not compressible, even a small change of volume upon rotation may lead to undesired effects like leakage, gas bubbles or loss of liquid to me delivered. As the gas in the gas volume is compressible and may change its volume upon positive and negative pressure, this volume change may compensate a volume change of the pump volume. The gas volume upon expansion may move the over-mold covering the gas reservoir toward the pump volume and vice versa. As the over-mold is elastic, the volume in the gas reservoir may vary.

According to an embodiment the gas reservoir has a volume between 1 mm\hat{ }3 and 20 mm\hat{ }3, in particular between 1 mm\hat{ }3 and 10 mm\hat{ }3, in particular 2 mm\hat{ }3+/−0.5 mm\hat{ }3.

Thus, a good balance between volume compensation on the one hand and form stability of the pump volume on the other hand can be achieved, which is required for pumping a medicament liquid.

According to an embodiment the first inlet and the first outlet end at the first inner wall portion of the pump volume of the pump chamber so that upon eccentrically rotating the pump rotor at least one of the apexes in rotation direction lies between the first inlet and the first outlet and that a rotational difference a between a rotational position of the pump rotor where an apex has just crossed the first inlet and a rotational position of the pump rotor where another apex just starts crossing the first outlet is not more than 14°, in particular not more than 10°, in particular between 6° and 10°.

Thus, it can be guaranteed that the outlet only opens, if it is ensured that the inlet is already closed, even as the worst coincidence of tolerances.

According to an embodiment the pump chamber has a trochoidal shape with said first lobe and a second lobe forming a second inner wall portion of a pump volume of the pump chamber with the pump rotor eccentrically rotating with the apexes/apex lines traveling along a trochoidal line corresponding to the trochoidal shape of the pump chamber, thus forming upon rotation a second suction volume and a second expelling volume.

Thus, a symmetrical buildup can be provided which avoids unbalance when operating the pump device and allows a proper sealing over the environment in order to avoid impurities. According to an embodiment the second inner wall portion has in a circumferential direction at least section-wise a notch formed therein forming upon rotation of the pump rotor a pressure compensation bypass between the second expelling volume and the second suction volume.

According to an embodiment the pump body at at least one axal side has a cover covering and sealing the pump chamber, wherein the cover in an area over the second lobe has a recess which is recessed in axial direction compared to an area over the first lobe, so that the outer dimensions of the pump rotor with the over-mold are larger than the corresponding inner dimensions of contacted portions of the pump chamber by a factor, and the factor in the area of the second lobe is smaller than the factor in the area of the first lobe.

Thus, the friction on the side of the second lobe can be reduced. As the second side may not be used for pumping, the sealing properties on the second lobe side may be lower, so that the oversize may be reduced by providing a recess.

According to an embodiment the recess recessed in axial direction in the area over the second lobe is between 0.01 mm and 0.2 mm, in particular between 0.05 mm and 0.1 mm, in particular 0.1 mm+/−0.03 mm. Alternatively, the recess may have a dimension which corresponds to a 50% pressure overlap.

Thus, a reliable reduction of friction can be achieved.

According to an embodiment an interface between the pump chamber and the cover provides a unique mounting position of the cover onto the pump chamber.

Thus, manufacturing and assembling can be made more reliable, as an unintended mix of orientation may be avoided during assembly.

According to an embodiment the interface is formed by a keyhole protrusion at one of the cover and the pump body housing which engages a keyhole at the other of the pump body housing and the cover, such that the cover can be placed over the pump chamber in only one position.

Thus, it can be achieved that the cover can be placed over the pump chamber in only one correct position or orientation.

According to an embodiment the cover is provided with an axially protruding rim protruding from the side of the cover facing the pump chamber.

Thus, a reliable connection and junction between the pump body housing and the pump body cover can be achieved.

According to an embodiment the second inner wall portion has in a circumferential direction at least section-wise a notch formed therein forming upon rotation of the pump rotor a pressure compensation bypass between the second expelling volume and the second suction volume.

Thus, upon use of only one lobe for pumping, the pressure build-up and under-pressure may be compensated. The purpose of the bypass in the second chamber area is to reduce the drive torque, and sealing defects driven by the generated air pressure in the second chamber. As the pressure upon rotation in the second suction chamber and the second expelling chamber are compensated, no second inlet and no second outlet have to be provided for the pressure compensation. Thus, the pump chamber may be sealed over the environment, which reduced impurities from outside.

According to an embodiment, the pump chamber in the pump volume of the second lobe has a second inlet in fluid communication with the in-port and a second outlet in fluid communication with the out-port with the pump rotor eccentrically rotating so that the three apexes of the rotor are in permanent sealing contact with the first inner wall portion of the first lobe and the second inner wall portion of the second lobe thus forming upon rotation a second suction volume in fluid communication to the second inlet and a second expelling volume in fluid communication to the second outlet.

Thus, a symmetrical pump geometry can be provided which provides two parallel operating pump sequences, one on the first lobe and the other on the second lobe. As the rotor is triangular and the pump chamber provides two lobes, the phase shift between the sucking an expelling characteristic of the first lobe is 180° over the sucking an expelling characteristic of the second lobe. When coupling both pump sequences together, the one thereof has a maximum expelling when the outlet is in the middle between two apexes, while the other one thereof has a minimum expelling when the third apex passes the outlet. A check valve at the outlet of each expelling chamber may avoid reflow of fluid into the expelling chamber. The same may apply for an inlet, and a check valve at the inlet of each suction chamber may avoid flow of fluid out of the suction chamber. It should be noted that the joint between the first lobe and the second lobe, i.e., where the first inner wall portion and the second inner wall portion transit into each other, is to be considered as belonging to both, the first lobe and the second lobe, and to both, the first inner wall portion and the second inner wall portion. It should also be noted, that in particular phases of the rotor rotation at least three distinct portions of the rotor are in permanent sealing contact for the first lobe and the at least three distinct portions of the rotor are in permanent sealing contact for the second lobe, which may share a portion, which may be a contact of the convex curved surface with the transit from the first lobe to the second lobe.

According to an embodiment, the second inlet is in fluid communication with the in-port and the second outlet is in fluid communication with the out-port.

Thus, not only first inlet, but also the second inlet can be connected to a common in-port, and also both, the first and second outlet is in fluid communication with the out-port so that the trochoidal medicament pump device can be operated with both lobes in parallel while having a single in-port and a single out-port, wherein the coupling of the parallel operated lobes and their inlets and outlets is established internally.

According to an embodiment, there is provided a giving set for providing a medicament from a reservoir to a patient, the giving set comprises a trochoidal medicament pump as described above, a first fluid conduit being with one end fixedly connected to the out-port and with the other end fixedly connected to a Luer-Lock.

Thus, the giving set can be provided with a medicament pump device without the need for additional joints between the pump device and the conduits. This may avoid impurities and keeps the possible number of connecting joints low. Further, it can be avoided that the medical pump device is unintentionally re-used, as the giving set definitively will not be re-used and disposed and so also the medical pump device. This makes the entire operation of the giving set safer.

According to an embodiment the first fluid conduit has an upstream section and a flexible downstream section, wherein the flexible downstream section is more flexible than the upstream section.

Thus, the giving set may stay more reliable connected to the patient. More flexible means that the more flexible section has a lower bending moment, in that the force required to bend to a certain extend along a given length is lower, in other words, more stable regarding bending. The flexible downstream section close to the patient allows compensation of a movement of the medicament pump device and unintentional separation of the giving set from the patient.

According to an embodiment, the first fluid conduit is made of a flexible tube of substantially constant diameter and wall thickness, wherein the flexible downstream section is formed by a pre-formed helical winding of the flexible tube, the geometry thereof providing higher flexibility than a non-helical portion.

Thus, it is possible to not only compensate a lateral, i.e., orthogonal to the extension of the conduit, movement of the medical pump device over the patient, but also a longitudinal, i.e., along the extension of the conduit, movement of the medical pump device over the patient. It should be noted that this may also include an embodiment, which has for the upstream portion a helical winding with low diameter windings, i.e., lower flexibility, and for the downstream portion a helical winding with large diameter windings, i.e., a higher flexibility.

According to an embodiment the giving set further comprises a second fluid conduit being with one end fixedly connected to the in-port and with the other end fixedly connected to a connector being adapted for being releasable connected to a fluid reservoir.

Thus, the giving set can be provided with a medicament pump device without the need for additional joints between the pump device and the conduits to the reservoir. This may avoid impurities and keeps the possible number of connecting joints low. Further, it can be avoided that the medical pump device is unintentionally re-used, as the giving set definitively will not be re-used and disposed and so also the medical pump device. This makes the entire operation of the giving set safer. It should be noted that the giving set may also a fixedly connected dripping volume between the reservoir and the medicament pump device.

According to an embodiment, the giving set further comprises a second fluid conduit being with one end fixedly connected to the in-port and with the other end fixedly connected to a pre-filled fluid reservoir.

Thus, a safe given set may be provided, which has a minimal number of connections and possible entering points for impurities. The medicament pump device may be adapted to the fluid in the pre-filled reservoir, so that the viscosity of the fluid matches the design of the medicament pump device. Any re-use can be avoided, which is of particular relevance for sensitive medicaments and critical diseases. The prefilled reservoir may include a predefined composition of medicament. A misalignment of medicament, in particular fluid and pump may be avoided.

According to an embodiment, the prefilled reservoir is gas volume free prefilled with a liquid.

Thus, the suction of gas can be avoided, and it can be guaranteed that regardless of the filling level of the reservoir always a fluid is delivered upon operation. As the trochoidal medicament pump device due to its construction is able to also suck against a certain height, the reservoir can also be disposed lower than the trochoidal medicament pump device. This makes it much easier to handle for a patient, in particular if the medicament pump device and the reservoir are used as a mobile delivery system which is carried by a patient and allows moving around.

According to an embodiment the giving set further comprises a tag representative for the liquid in the pre-filled reservoir and being adapted to be read out by a tag reader of a medicament pump driving device.

Thus, the operation of the trochoidal medicament pump device may be adapted to the type of fluid or liquid in the reservoir. Some liquids may have particular properties, which allow a defined driving dynamic, which can be setup based on the read-out tag, so that not only the medicament pump may be adapted to the liquid, but also the operation, e.g., the revolutions or change rate of revolutions, of the medicament pump device. This may of particular relevance if the liquid is a or includes a non-Newtonian fluid.

It should be noted that the above-described embodiments may also be combined and in a combined form provide a synergetic technical effect and synergetic benefits which go beyond the sum of the single technical effects and benefits.

### BRIEF DESCRIPTION OF THE FIGURES

The invention will be described by way of the following drawings:

FIG. 1: illustrates a schematic build-up in a top view of a trochoidal medicament pump device according to an exemplary embodiment.

FIG. 2: illustrates a rotor core for a pump rotor for use in a trochoidal medicament pump device according to an exemplary embodiment.

FIG. 3: illustrates a pump rotor with an over-molded core for use in a trochoidal medicament pump device according to an exemplary embodiment.

FIG. 4: illustrates a top view of a trochoidal medicament pump device with removed cover according to an exemplary embodiment.

FIG. 4A illustrates a rotor and pump body in a particular position with respect to each other according to an exemplary embodiment.

FIG. 5: illustrates an exploded view of a trochoidal medicament pump device without cover according to another exemplary embodiment.

FIG. 5A illustrates a cover/lid for covering the pump chamber according to an exemplary embodiment.

FIG. 6: illustrates a schematic build-up in a top view of a trochoidal medicament pump device according to another exemplary embodiment.

FIG. 7: illustrates giving set with a connected liquid/fluid reservoir and a trochoidal medicament pump device according to an exemplary embodiment.

FIG. 8: illustrates giving set with a connected liquid/fluid reservoir and a trochoidal medicament pump device connected to driving device according to an exemplary embodiment.

FIG. 9: illustrates giving set with a fluid conduit to be connected to a patient and a fluid conduit to be connected to a liquid reservoir according to an exemplary embodiment.

It should be noted that same or similar reference numerals illustrate same or similar components. Along these Figures exemplary embodiments of the invention will be described as follows.

### DETAILED DESCRIPTION OF EXEMPLARY EMBODIMENTS

The invention will be described along exemplary embodiments, which are illustrated in the above references figures and will be described in detail in the following.

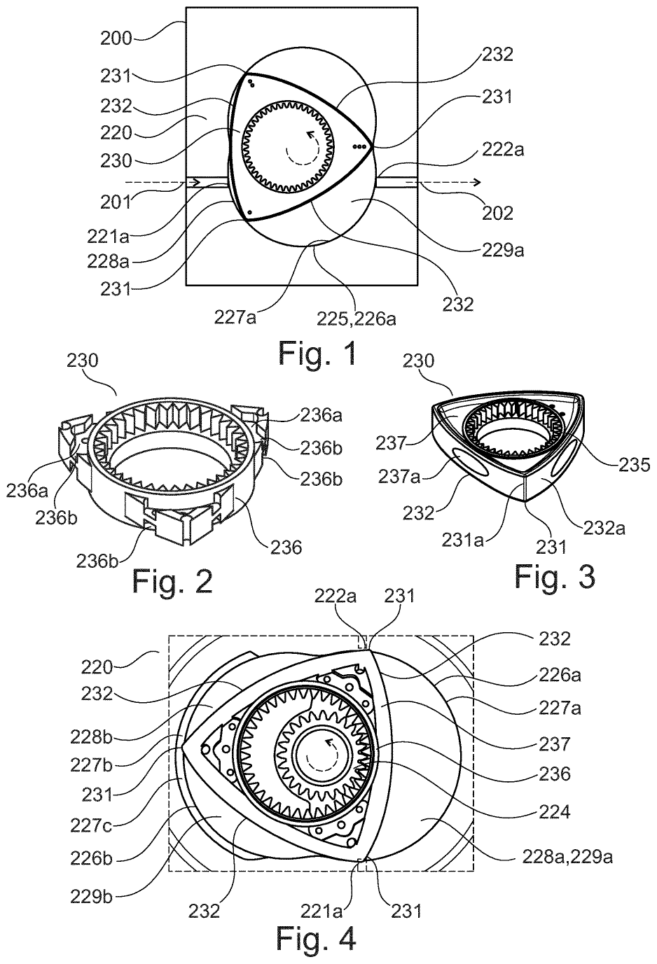

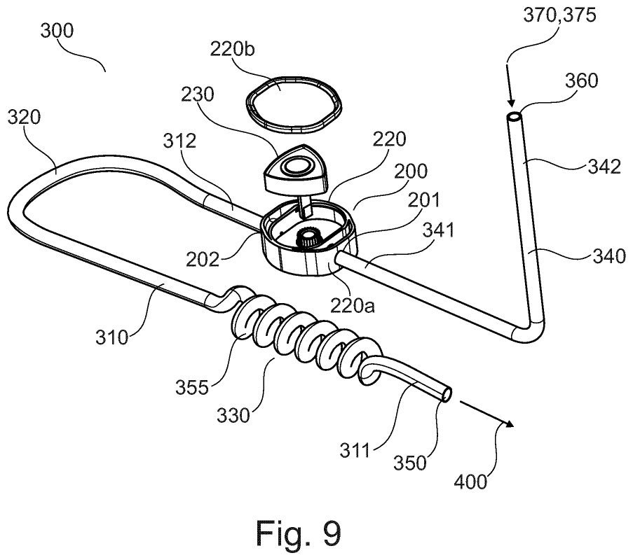

FIG. 1 illustrates a schematic build-up in a top view of a trochoidal medicament pump device according to an exemplary embodiment. The trochoidal medicament pump device 200 has an in-port 201 and an out-port 202. The in-port receives the fluid or liquid to be pumped and the out-port delivers the fluid or liquid to be delivered. The in-port and the out-port may have releasable couplings or may be fixedly connected to conduits or the like. The trochoidal medicament pump device 200 has a pump body 220, which may have a housing 220a and a cover 220b. The housing 220a and the cover 220b, which is illustrated in FIG. 9, define within the pump body 220 a pump chamber 225. The pump chamber 225 has rotating therein a pump rotor 230. The pump rotor 230 is of a triangular shape when seen from top. Seen from top the triangular shape has three apexes 231 and between each two apexes a convex curved line 232. When seen as a three-dimensional object, the apexes 231 are apex lines 231a extending parallel to the rotational axis of the pump rotor 230, and the convex curved lines 232 are convex curved surfaces 232a, extending between the apex lines 231a. The apex lines 231a and the convex curved lines 232a are in contact with wall portions 227a, 227b of the pump chamber 225. The apex lines 231a are always in contact with one of the wall portions 227a, 227b. The pump chamber 225 has two lobes 226a, 226b. Within the pump chamber 225 the pump rotor 230 rotates, so that the contact between the pump rotor 230, in particular its apex lines 231a and also the convex curved surfaces 232a, and the wall sections 227a, 227b of the lobes 226a, 226b form chambers, which rotate with the eccentrically rotating pump rotor 230 and successively are brought into communication with an inlet 221a and an outlet 222a. The apexes 231 of the eccentrically rotating pump rotor 230 trace a trochoidal line corresponding to a trochoidal shape that is equivalent or larger than the corresponding inner dimensions of the pump chamber 225. When in connection with the, or one of the inlets 221a, the chamber operates as a suction chamber 228a. As the chamber upon rotation successively enlarges its volume, the chamber sucks a fluid or liquid from the in-port 201. Upon rotation, the suction chamber rotates until it leaves the inlet 221a. Afterwards, the chamber gets into communication with an outlet 222a and becomes an expelling chamber 229a. Upon further rotation, the expelling chamber 229a successively decreases its volume and thus expels the fluid through the output 222a until the volume arrives at its minimum, which may be close to zero. At the same time, the following geometry of the rotor 230 proceeds likewise, so that the next chamber becomes the expelling chamber 229a and expels the next fluid amount upon rotation of the pump rotor 230. Upon eccentrical rotation of the rotor 230 the first inlet 221a after filling the volume with a liquid to be pumped will be closed by one of the apexes 231 so that the liquid can be fed to the first outlet 222a. Between closing the volume, i.e., closing the first inlet 221a, and opening again the volume, i.e., opening the first outlet 222a there is a rotational difference a between a rotational position of the pump rotor 230 where an apex has just crossed the first inlet 221a and a rotational position of the pump rotor 230 where another apex 231 just starts crossing the first outlet 222a. This is a kind of overlap during which the volume is closed. As the volume due to the shape of the trochoidal shape of the lobes may vary, this overlap may be kept low, but still guaranteeing that the volume remains closed. This overlap may be not more than 14°, in particular not more than 10°, in particular between 6° and 10°.

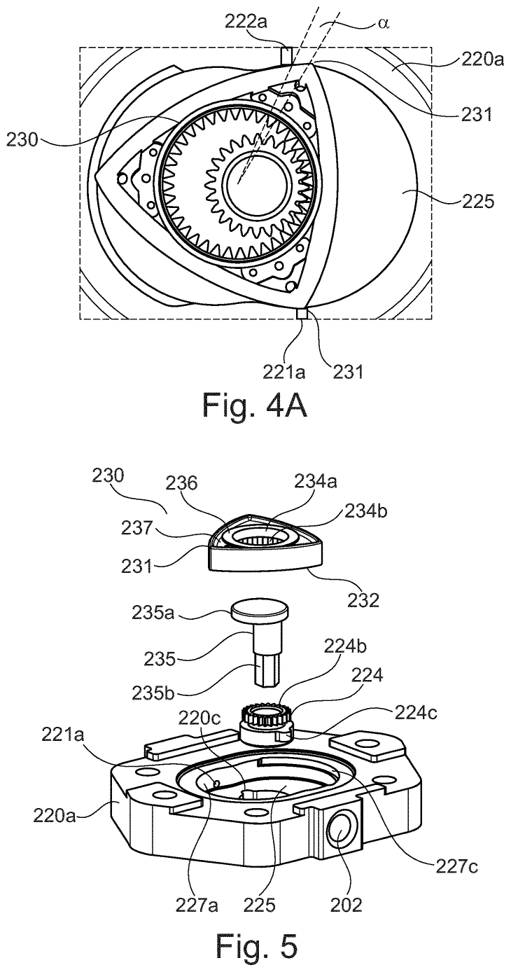

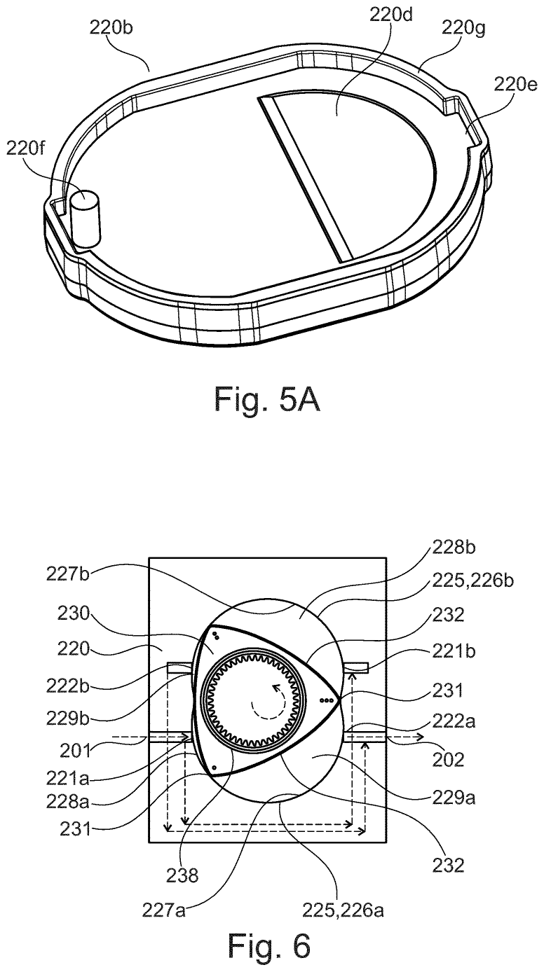

FIG. 2: illustrates a rotor core 236 for a pump rotor 230 for use in a trochoidal medicament pump device 200 according to an exemplary embodiment. The rotor core forms a stable structure and provides the rigid geometry for driving the pump rotor 230 and to keep it in the correct position and trajectory upon eccentric rotation. For this purpose, the pump rotor 230, in particular the rigid core 236 has a sliding or guiding surface 234a on the pump rotor 230 and in particular the rigid rotor core 236 of the pump rotor 230, which receives an eccentrically traveling disk 235a of a driving pinion 235, which eccentrically rotation disk 235a forces the pump rotor 230 onto an eccentric trajectory. The pump rotor 230, in particular its rigid core 236 has also a gear section 234b, which meshes with a gear section 224b of a gear wheel 224 on the pump body 220, in particular the housing 220a, and upon eccentric traveling, forces the pump rotor 230 to rotate. The meshing can be seen in FIG. 4, where the pinion 235 and the eccentrically rotation disk 235a thereof being removed. The design of the driving pinion 235 can be seen in FIG. 5. The rigid core 236 may have an undercut geometry 236a, which allows the over-mold 237 to be connected reliably to the rigid core. According to an embodiment the rigid rotor core has an undercut geometry 236a, wherein the undercut geometry 236a along each a convex curved line 232 has an undercut orthogonal to the rotational axis of the rotor. The rigid rotor core 236 along each a convex curved line 232 may have a plurality of recesses 236b being adapted for providing access for an over-mold to the undercut geometry 236a. The core 236 with the undercut geometry offers a maximized surface for e.g. chemical bonding like gluing as well as a maximized undercut with narrow openings so that the over-mold elastic material 237 cannot slip back through the slot even under high pressure and high tensile forces applied to the over-mold material. Smaller recesses 236b and wider recesses 236b may be provided providing a safe mechanical fixation of the elastic material, e.g., LSR. The dimension of the slots, i.e., a gap width, depends on the elasticity and shear force of the elastic over-mold material. Cylindrical holes parallel to the rotation axis may be provided for fixing top portions and bottom portions of the over-mold under the undercut geometry. The dimensions of these holes may depend on the flow index of the unset over-mold material and the shear forces.

FIG. 3 illustrates a pump rotor with an over-molded core for use in a trochoidal medicament pump device according to an exemplary embodiment. The core 236 serves for a stable and defined geometry for driving the pump rotor 230. The elastic or elastomeric over-mold 237 serves for sealing the pump rotor 230 over the pump chamber 225, in particular for sealing over the wall section 227a, 227b and the bottom of the housing 220a and the cover 220b of the housing. The apex lines 231a seal over the wall sections 227a, 227b, whereas the convex curved lines 232 seal over the bottom of the housing 220a and the cover 220b, as illustrated in FIG. 5. The pump rotor 230 with the core 236 and the over-mold 237 may be manufactured in a 2K-manufacturing process. The core may be made of e.g., reinforced Polyetheretherketon PEEK or reinforced Polyethylene terephthalate PET. The core may also be made from other materials being sufficiently rigid, like PBT. The rotor core may be made of a material filled with reinforcing fibers for a higher stability. The rotor core may be made of a material including PBT and 30+/−10 weight % glass fibers. Instead of glass fibers also carbon fibers may be used. The aforementioned materials for the core revealed a good compatibility with the over-mold material. The over-mold 237 may be made of liquid silicone rubber LSR, ethylene propylene diene monomer rubber EPDM, thermoplastic elastomer TPE, thermoplastic polyurethane TPU, butyl rubbers and nitrile rubbers. The aforementioned materials have a good compatibility with the selected materials of the core 236, but also with the materials of the pump body 220, in particular the housing 220a and the cover 220b. The compatibility with the pump body 220 includes a compatibility with respect to resistivity over medicaments and compatibility with the viscosity of the medicaments, as well as friction between the over-mold 237 and the materials of the pump body 225. It should be noted that this also applies for a pump body 225, which has a two or more material composition with particular materials for the pump body 225 and other materials for the wall sections 227a, 227b, the bottom and the cover 220b thereof, in other words those parts of the body which are in contact with the pump rotor 230, in particular its over-mold 237. The over-mold 237 may be made of a biocompatible material, which does not degenerate or release substances when coming into contact with biological material or medicaments. Also the pump body 225 or at least those parts facing the pump volume may be made of a biocompatible material, which does not degenerate or release substances when coming into contact with biological material or medicaments. The over-mold 237 may include a gas reservoir or blister 237a for a volume compensation of the volume covered by the rotor 230 and the pump chamber 225. Upon rotation of the rotor 230, the covered volume may vary due to the trochoidal inner shape of the pump chamber. As a pumped liquid or fluid usually has a low compressibility, it may be desired to compensate volume variations in order to avoid larger over pressure or under pressure at the pumped liquid or fluid. The gas reservoir, when covered or surrounded by the LSR may expand or compress upon volume change and allow the LSR face between the apexes to expand or compress, so that the gas reservoir may compensate the volume change without applying larger over pressure or under pressure to the pumped liquid or fluid. The gas reservoir 237a may be totally covered by the LSR as a blister. The gas reservoir 237a may also be enclosed between a face of the core 236 and a face of the over-mold 237. The larger the gas volume compared to the volume variation, the lower is the over or under pressure applied to the liquid or fluid. It should be noted that each section between two apexes may have its own hermetically closed gas reservoir 237a. It should be noted that the tree gas reservoirs 237a may also be connected to each other by conduits or tubes, so that the entire resulting gas reservoir may be shared.

FIG. 4 illustrates a top view of a trochoidal medicament pump device 200 with removed cover according to an exemplary embodiment. FIG. 4 illustrates the trochoidal medicament pump device with removed cover 220b and with removed driving pinion 235. Upon rotation of the pump rotor 230 in a planetary gear manner around the gear wheel 224, which is fixed to the pump body 220, the apexes 231 of the pump rotor 230 follow the trochoidal shape of the pump chamber 225, in particular its wall portion 227a on the right side in FIG. 4. The apex lines 231a seal the pump rotor 230 over the inner wall portions and this form the suction chamber 228a and the expelling chamber 229a, respectively. The fluid to be pumped and delivered enters the pump chamber 225 over the inlet 221a and leaves the pump chamber 225 over the outlet 222a. The illustrated situation in FIG. 4 is the transit where the suction chamber 228a becomes an expelling chamber 229a. It can be seen, that one of the apexes 231 has just passed the inlet 221a and thus closed the chamber and terminated the suction phase, before the ahead traveling apex 231 has not yet passed the outlet 222a and thus not yet opened the chamber for expelling the fluid. It should be noted that the apexes 231 also follow the wall sections 227b on the other side, the left side in FIG. 4. However, in the illustrated embodiment of FIG. 4, which corresponds to the embodiment illustrated in FIG. 1, only one inlet 221a and only one outlet 222a are provided on the right lobe 226a.

The other lobe 226b on the left side does not have an inlet and an outlet, so that in the respective the suction chamber 228b on the left side, a vacuum arises upon rotation. At the same time in the expelling chamber 229b on the left side the pressure arises upon rotation. As the vacuum in chamber 228b and the over pressure in chamber 229b lead to increased friction and load of the rotor 230, this vacuum and pressure, respectively should be avoided. As a further inlet and outlet should be avoided in case they are not used for pump and delivery, the pressure difference is compensated through a notch 227c, which follows along a particular section of the wall section 227b on the left side in FIG. 4. The notch bypasses the sealing of the apex 231 at the inner wall portion 227b and allows the overpressure from the expelling chamber to transit and compensate the vacuum in the suction chamber 228b. Thus, the pressure difference can be compensated, and the friction and load can be reduced. The notch may be formed by a chamfer as illustrated in FIG. 4. The chamfer may have rounded edges at least at the side where the apex line is in contact with the edge of the chamfer. The notch may also be realized by a groove along the inner wall portion 227b or even by a bypassing channel having one opening in the area of the suction chamber 228b and the other opening in the area of the expelling chamber 229b.

FIG. 4A illustrates a rotor 230 and a pump body 220 in a particular position with respect to each other. From FIG. 4A it can be seen that upon rotation at least one apex 231 lies between the first inlet 221a and the first outlet 222a end at the first inner wall portion 227a of the pump volume of the pump chamber 225. A rotational difference a (alpha) between a rotational position of the pump rotor 230 where an apex has just crossed the first inlet 221a and a rotational position of the pump rotor 230 where another apex 231 just starts crossing the first outlet 222a is not more than 12°, in particular not more than 10°, in particular between 6° and 10°. This overlap ensures that the inlet always is closed before the outlet opens in order to avoid flow back of the liquid to be pumped. As manufacturing leads to particular tolerances, the overlap has a dimension so that even in the worst coincidence of the manufacturing measures it can be ensured that the inlet is always closed before the outlet opens.

FIG. 5 illustrates an exploded view of a trochoidal medicament pump device without cover 220b according to another exemplary embodiment. The position of the cover 220b is illustrated in FIG. 9. It should be noted that the traveling trajectory of the pump rotor 230 is defined by the eccentric disk 235a, which is illustrated in FIG. 5, and which is omitted in FIG. 4 for illustrative purposes. The eccentrically rotating disk 235a upon centric rotation of the pinion 235 and its drive coupling 235b forces the pump rotor 230 onto an eccentric path. The eccentric disk 235a is rotationally bedded over a corresponding bearing surface 234a on the rotor 230, in particular its core 236. As during eccentric rotation, the gear section 234b of the pump rotor 230 remains in engagement with the gear section 224b of gear wheel 224 of the pump body, the pump rotor 230 not only eccentrically travels along an eccentric path, but also rotates, so that the apexes follow the trochoidal path of the first and second lobe 226a, 226b and the corresponding wall portions 227a, 227b. As can be seen in FIG. 5, the gear wheel 224 is provided as a separate element which however has a protrusion 224c which engages a detention 220c in the pump body so that the gear wheel 224 is fixed over the pump body. The fixation can also be realized by other measures, like adhesives, positive fit connections or by providing the gear wheel 224 in one piece with the pump body 225. With respect to the remaining elements in FIG. 5 it is referred to the above-described Figures.

The pump rotor 230 has an over-mold 237, which has with respect to the contacting portions to the wall portions 227a, 227b of the pump chamber 225 a larger dimension than the pump chamber, so that the elastic or elastomeric over-mold is compressed and provides a reliable sealing. These contacting portions are the apex lines 231a of the rotor 230 and also portions of the convex curved surfaces 232a, which are in contact to the inner wall portions 227a, 227b, in particular with the transit from the first wall portion 227a of the first lobe 226a to the second wall portion 227b of the second lobe 226b. The elastic or elastomeric material of the over-mold 237 may be selected out of a first material class, the class may be a group consisting of liquid silicone rubber LSR, ethylene propylene diene monomer rubber EPDM, thermoplastic elastomer TPE, thermoplastic polyurethane TPU butyl rubbers and nitrile rubbers. In particular, if the over-mold 237 is made of liquid silicone rubber LSR, the LSR is designed to have a hardness between 10 and 100 Shore (ShA), in particular between 50 and 100 Shore (ShA), in particular between 60 and 70 Shore (ShA). The hardness in some instances depend on the expected pressure of the fluid and of the viscosity of the fluid to be pumped. As an alternative, the over-mold may be made of a medical grade TPE (thermoplastic elastomer), and the TPE over-mold 237 is designed to have a hardness between 29 and 90 Shore (ShA), in particular between 40 and 80 Shore (ShA), in particular between 50 and 70 Shore (ShA). As a further alternative, the over-mold may be made of a TPU (thermoplastic Polyurethane), and the TPU over-mold 237 is designed to have a hardness between 29 and 90 Shore (ShA), in particular between 40 and 80 Shore (ShA), in particular between 50 and 70 Shore (ShA). The hardness in some instances depend on the expected pressure of the fluid and of the viscosity of the fluid to be pumped.

LSR may have a hardness Shore A of 50 to 60 and a density between 1.1 g/cm\hat{ }3 and 1.2 g/cm\hat{ }3. LSR may be a self-lubricating LSR which controllably releases lubricant. The self-lubricating LSR may be a biocompatible LSR.

LSR may be Silopren® LSR 4655 SL of Momentive in the specification of 07.04.2016 with hardness Shore A of 55 according to DIN 53 505, a density of 1.13 g/cm\hat{ }3 according to DIN 53 479 A, a tensile strength of 8 N/mm\hat{ }2 according to DIN 53 504 S2, an elongation at break of 450% according to DIN 53 504 S2 and a tear strength of 45 N/mm according to ASTM D 624 B.

LSR may be SILPURAN® 6600/60 A/B of Wacker in the specification of 11.11.2022 with hardness Shore A of 60 according to DIN ISO 48-4, a density of 1.12 g/cm\hat{ }3 according to DIN EN ISO 1183-1 A, a tensile strength of 9.0 N/mm\hat{ }2 according to ISO 37 type 1, an elongation at break of 310% according to ISO 37 type 1 and a tear strength of 26 N/mm according to ASTM D 624 B.

LSR may be SILPURAN® 6760/50 A/B of Wacker in the specification of 11.11.2022 with hardness Shore A of 50 according to DIN ISO 48-4, a density of 1.10 g/cm\hat{ }3 according to DIN EN ISO 1183-1 A, a tensile strength of 8.4 N/mm\hat{ }2 according to ISO 37 type 1, an elongation at break of 600% according to ISO 37 type 1 and a tear strength of 27 N/mm according to ASTM D 624 B.

The self-lubricating LSR may be adapted to allow at least 6000 revolutions of the rotor 230 within the pump body 220. For some applications the self-lubricating LSR may be adapted to allow between 1500 and 6000 revolutions of the rotor 230 within the pump body 220, in particular between 3000 and 4000 revolutions.

It revealed that a considerable number of infusions are based on fluids/liquids having a viscosity, which is similar to the viscosity of water, which is for water at 20 °C about 1 cP at a flow of 500 ml/h. When using a small canula, e.g., that of a 31 G needle with an inner diameter of 0.11 mm, an exemplary inner pressure within an exemplary pump chamber is between 6 and 7 bar.

The hardness may also depend on the material used for the pump body 225, in particular for the inner wall sections 227a, 227b, the bottom and the cover 220b of the pump body 220. These sections may be manufactured of a material of a second class of material, wherein the second material class is a group consisting of polyethylene terephthalate PET, polycarbonate PC, Tritan, high density polyethylene HDPE, cycloolefin copolymer COC, PBT, PPS, and PEI. In case Tritan is used, the biocompatible material may be a Tritan compounded with a medical grade absorbent. The absorbent is an optical absorbent absorbing an optical radiation of a particular wavelength or wavelength spectrum. The absorbent in particular may absorb a wavelength between 960 nm and 1000 nm, in particular a wavelength of 980 nm+/−10 nm. The absorbent is in particular adapted for absorbing an optical radiation of a welding laser, so that the light of the laser is absorbed and efficiently converted in a material heat and melt for welding purposes. The inner dimensions of the pump chamber 225, in particular the inner wall portions 227a, 227b, and the outer dimensions of the pump rotor 230, in particular its over-mold 237 may also depend on the expected pressure and the expected viscosity of the fluid. The outer dimensions of the pump rotor 230 with the over-mold 237 may be larger than the corresponding inner dimensions of contacted portions of the pump chamber 225 by a factor, which factor is between 1.01 and 1.1, in particular between 1.01 and 1.05, in particular between 1.01 and 1.03. This means the oversize by this factor has to be compensated by the elastic or elastomeric material of the over-mold 237. As the over-mold has a smaller dimension than the entire rotor 230, and the core 236 is not or only less elastic or elastomeric, the elastic or elastomeric material of the over-mold needs to be more elastic or elastomeric than by only the above-mentioned factor. The elasticity may occur in different directions, i.e. in radial direction and in axial direction. The reason is that the acting frictional forces have different directions, and the sealing along the apex line 231a is shorter than the convex curved sealing line 232. Further, the movement of the rotor 230 results in a traverse force onto the apex line 231a, whereas the force onto the convex curved lines 232 is at least partially along the extension of the convey curved sealing line. Therefore, the factor in the radial direction may be between 1.01 and 1.1 (i.e., percentage increase 1-10%), in particular between 1.01 and 1.05 (i.e., percentage increase 1-5%), in particular between 1.02 and 1.03 (i.e., percentage increase 2-3%) whereas the factor in the axial direction may be between 1.01 and 1.1 (i.e., percentage increase 1-10%), in particular between 1.01 and 1.05 (i.e., percentage increase 1-5%), in particular between 1.01 and 1.03 (i.e., percentage increase 1-3%). The relation of the percentage increase of the radial factor and the percentage increase of the axial factor (percentage increase axial factor divided by the percentage increase radial factor) may be between 1 and 10 (i.e., between 1%/1% and 10%/1%), in particular between 1 and 5 (i.e., between 1%/1% and 5%/1%), in particular between 2 and 3 (i.e., between 2%/1% and 3%/1%).

FIG. 5A illustrates a cover/lid 220b for covering the pump chamber according to an exemplary embodiment. The cover 220b covers and seals the pump chamber 225. In case the first lobe is used as pumping volume and the second lobe is not used as a pumping volume, the cover 220b in an area over the second lobe 226b may have a recess 220d or recessed area which is recessed in axial direction compared to an area over the first lobe 226a. The outer dimensions of the pump rotor 230 with the over-mold in this area may remain larger than the corresponding inner dimensions of contacted portions of the pump chamber 225, so that the sealing remains. The factor by which the outer dimensions of the pump rotor 230 with the over-mold in this area is larger than the corresponding inner dimensions of contacted portions of the pump chamber 225, however is smaller than the factor in the area of the first lobe 226a. Thus, the friction in the area of the second lobe may be lower than the friction in the area of the first lobe. As the pumping only takes place in the first lobe, the oversize factor leading to a sealing is required in the area of the first lobe, but not in the second lobe. It should be noted that the dimensions of the cover 220b and its recess 220d bay be designed so that in the area of the second lobe no oversize factor exists leading to a further reduction of friction. In case the friction is lowered in the second lobe, also the entire friction is reduced, leading to reduced driving forces and more exact delivery of liquids by the pump. The recess 220d recessed in axial direction in the area over the second lobe 226b may be between 0.01 mm and 0.2 mm, in particular between 0.05 mm and 0.15 mm, in particular 0.1 mm+/−0.04 mm. An interface 220e, 220f between the pump chamber 225 and the cover 220b may provide a unique mounting position of the cover 220b onto the pump chamber 225. The interface may be formed by a unique outer shape of the cover and/or by a keyhole protrusion 220f at the cover 220b or the counterpart which engages a keyhole 220e at a side of the pump chamber 225 or the cover, respectively, such that the cover 220b can be placed over the pump chamber 225 in only one position. The cover 220b may be provided with an axially protruding rim 220g protruding from the side of the cover facing the pump chamber 225 for easier and more reliably assembly of the cover.

FIG. 6 illustrates a schematic build-up in a top view of a trochoidal medicament pump device according to another exemplary embodiment. The trochoidal medicament pump device 200 as illustrated in FIG. 6 is similar to the device illustrated in FIG. 1. In addition to what is described with respect to FIG. 1, the trochoidal medicament pump device 200 of FIG. 6 has not only an inlet 221a and an outlet 222a in the first lobe 226a, but also has a further inlet 221b and a further outlet 222b in the second lobe 226b. This allows using also the second lobe as a pump, which may be operated in parallel to the pump formed in the first lobe 226a. The first inlet 221a and the second inlet 221b both may be in fluid communication with the in-port 201. At the same time the first outlet 222a and the second outlet 222b may be in fluid communication with the out-port 202. Thus, both pump paths can be coupled via a common in-port 201 and a common out-port 202. The in-port 201 receives the fluid or liquid to be pumped and the out-port 202 delivers the fluid or liquid to be delivered. The in-port and the out-port may have releasable couplings or may be fixedly connected to conduits or the like. The triangular pump rotor 230 travels as describe above. The apex lines 231a and the convex curved lines 232a are in contact with wall portions 227a, 227b of the pump chamber 225. The apex lines 231a are always in contact with one of the wall portions 227a, 227b. The pump chamber 225 has two lobes 226a, 226b. Within the pump chamber 225 the pump rotor 230 rotates, so that the contact between the pump rotor 230, in particular its apex lines 231a and also the convex curved surfaces 232a, and the wall sections 227a, 227b of the lobes 226a, 226b form chambers, which rotate with the eccentrically rotating pump rotor 230 and successively are brought into communication with the first inlet 221a, the first outlet 222a.

the second inlet 221b and the second outlet 222b. When in connection with the respective inlet 221a, 221b, the chamber operates as a suction chamber 228a, 228b. As the chamber upon rotation successively enlarges its volume, the chamber sucks a fluid or liquid from the in-port 201. Upon rotation, the suction chamber rotates until it leaves the respective inlet 221a, 221b. Afterwards, the chamber gets into communication with the respective outlet 222a, 222b and becomes an expelling chamber 229a, 229b. Upon further rotation, the expelling chamber 229a, 229b successively decreases its volume and thus expels the fluid through the output 222a, 222b until the volume arrives at its minimum, which may be close to zero. At the same time, the following geometry of the rotor 230 proceeds likewise, so that the next chamber becomes the expelling chamber 229a, 229b and expels the next fluid amount upon rotation of the pump rotor 230. The in-flow follows in FIG. 6 the dashed lines and arrows, while the out flow follows the dashed-dotted lines in FIG. 6.

FIG. 6 further illustrates that the rotor 236 may have an internal sealing lip 238 which is provided on a at least one bottom or top side of the rotor 236 being oriented toward a cover, wherein the internal sealing lip 238 may seal over a top or bottom cover. The internal sealing lip 238 is provided on the respective side radially between a gear section 234b of the rotor 236 and the convex curved lines 232 and forms additionally to a main sealing line along the convex curved lines, a further backup seal. The internal sealing lip 238 may be set back compared to the main seal so that it has a lower friction than the main seal along the convex curved lines. The internal seal lip 238 may serve as a safety seal to prevent possible air or dirt entry through the eccentric position opening. This lip may be arranged cylindrically around the eccentric bearing, set back compared to the main seal with less pressure on the housing. The internal sealing lip may be made from the same material as the remaining over-mold and manufactured in the same injection molding process step and may be connected through channels through which the material flows during injection molding.

As can be seen from FIG. 6, the first suction chamber 228a is at a minimum volume starting the suction process, while the second suction chamber 228b is at a maximum volume having almost finished the suction process. Likewise, the first expelling chamber 229a is at a maximum volume starting the expelling process, while the second expelling chamber 229b is at a minimum volume having almost finished the expelling process. As the expelling process is not linear and has a break while an apex crosses the respective output, a single pump has a non-continuous feeding upon continuous rotation of the pump rotor 230. However, as the expelling process of the pump in the first lobe 226a is phase shifted over the expelling process in the second lobe 226b, the interruption may be compensated. So that no interruption occurs at the out-port 202. In order to avoid unintended backflow of fluid into a chamber, check valves or flow resistors (not illustrated in FIG. 6) may be provided which avoid or reduce unintended back flow.

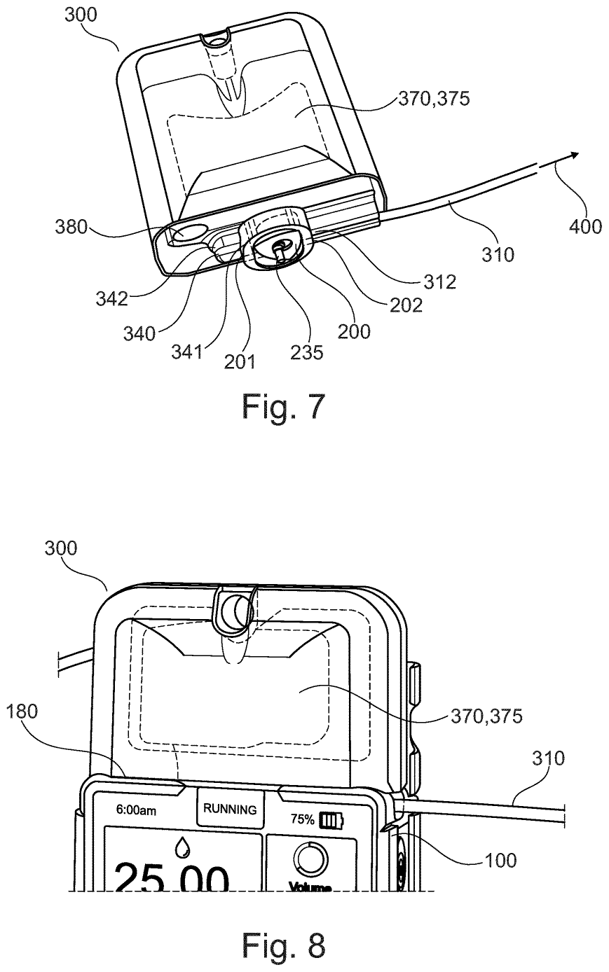

FIG. 7 illustrates a giving set with a connected liquid/fluid reservoir and a trochoidal medicament pump device according to an exemplary embodiment. The trochoidal medicament pump device is as described above with respect to FIGS. 1 to 6. A giving set is a device which is used for applying a medicament to a patient. Conventional giving sets are known and may include a connection to a reservoir and a connection to a patient and dosing facilities for setting a flow rate. The giving set 300 includes a trochoidal medicament pump device 200 and further has a first fluid conduit 310 for connecting the trochoidal medicament pump device 200, in particular its out-port 202 to a patient 400. Likewise, the giving set 300 has a second fluid conduit 340 for connecting the trochoidal medicament pump device 200, in particular its in-port 201 with a reservoir 370. The giving set 300 illustrated in FIG. 7 has the reservoir 370 fixedly connected to the second fluid conduit 340, The first end 341 of the second conduit 340 is fixedly connected to the in-port 201 of the trochoidal medicament pump device 200 and the second end 342 of the second conduit 340 is fixedly connected to the reservoir 370. This reduces joining connections and impurities in the system. The reservoir 370 may be a pre-filled reservoir 375, so that the giving set is ready for use in combination with a trochoidal medicament pump device 200. The first conduit 310 with its first end can be connected to the patient 400, and with its second end 312 is fixedly connected to the out-port 202 of the trochoidal medicament pump device 200. The trochoidal medicament pump device 200 may be adapted to the fluid in the prefilled reservoir 375. As the reservoir usually is not refilled and disposed, also the trochoidal medicament pump device 200 is disposed and an unintended re-use can be avoided. The reservoir may be a flexible volume like a bag and with the fluid therein may be gas free, so that irrespective of the position of the reservoir and its outlets, a fluid can be taken from the reservoir. As the trochoidal medicament pump device 200 is able to suck, the reservoir may even be disposed lower than the trochoidal medicament pump device 200, i.e., with the trochoidal medicament pump device 200 on top of the reservoir 370, 375. The giving set 300 may have a readable tag 380 carrying information representative for the giving set, e.g., with respect to the tape of fluid, the volume of the reservoir, the type of the trochoidal medicament pump device 200, etc. This tag can read by a tag reader 180 on e.g., a driving device 100 for the trochoidal medicament pump device 200, as will be described with respect to FIG. 8. The tag 380 may be an RFID device, or a machine-readable optical tag, but may also be an actively sending tag. The tag may also include a certain intelligence and may be connected to sensors or the like, which sense parameters on or of the giving set, so that this information can be provided upon reading the tag. The trochoidal medicament pump device 200 has a coupling to be driven by a driving device 100. The coupling may be realized by a coupling portion 235 of a driving pinion, as describes above, and may be coupled to the driving device 100 by the coupling portion 235.

FIG. 8 illustrates a giving set with a connected liquid/fluid reservoir and a trochoidal medicament pump device connected to driving device according to an exemplary embodiment. The giving set 300 is coupled via the above-described driving coupling 235 to the driving unit 100. The driving unit may also have a tag reading facility 180 for reading the tag 380 or communicating with the tag 380. The trochoidal medicament pump device 200 in this embodiment is received in aby of the driving unit in order to protect the trochoidal medicament pump device 200, its ports 201, 202 and also the conduits 310, 340. The giving set 300 may be locked with respect to the driving unit 100 in order to avoid unintended separation of the reservoir 370, 375 and the trochoidal medicament pump device 200 from the driving unit.

FIG. 9 illustrates giving set with a fluid conduit to be connected to a patient and a fluid conduit to be connected to a liquid reservoir according to an exemplary embodiment. FIG. 9 illustrates the implementation of a trochoidal medicament pump device as described above with respect to FIGS. 1 to 6. The giving set 300 includes the trochoidal medicament pump device 200 and further has a first fluid conduit 310 for connecting the trochoidal medicament pump device 200, in particular its out-port 202 to a patient 400. Likewise, the giving set 300 has a second fluid conduit 340 for connecting the trochoidal medicament pump device 200. For this purpose, the second conduit 340 has a first end 341, which is connected to the trochoidal medicament pump device 200, in particular its in-port 201. A second end 342 of the second conduit 340 may be connected to a reservoir 370, 375. This can be done via a connector 360. The reservoir 370, 375 may also be fixedly connected as described with respect to FIG. 7 above. The giving set illustrated in FIG. 9 for the first conduit 310 has an upstream section 320 and a downstream section 330. In the upstream section 320 the second end 312 of the first conduit 310 is fixedly connected to the out-port 202 of the trochoidal medicament pump device 200. In the downstream section 330 the first end 311 of the first conduit 310 is connectable to a patient 400, e.g., via a Luer lock device 350. The downstream section 330 in this embodiment has a higher flexibility than the upstream section, which means that it can be easier bended. This is achieved in the illustrated embodiment by providing a helical winding 335, which elongates the flow length of the conduit and therefore allows higher flexibility, however without elongated dimensions, as the helical winding 335 is not longer than a comparable other section and does not increase the physical distance from the reservoir to the patient. The helical winding may not only compensate bending forces traverse to the longitudinal extension, but also forces along the longitudinal extension of the first conduit, as the helical winding may act like a spring.

### REFERENCE NUMBERS

100 driving unit for driving the trochoidal medicament pump device
180 tag reader on driving unit
200 trochoidal medicament pump device
201 in-port of trochoidal medicament pump device
202 out-port of trochoidal medicament pump device
220 pump body
220a housing of pump body
220b cover of pump body
220c locking notch/detention of pump housing/pump body
220d recess in cover
220e keyhole for receiving key protrusion for unique positioning of cover
220f key protrusion for engaging keyhole for unique positioning of cover
220g protruding (circumferencing) rim at cover for assembling
221a first inlet of pump chamber
221b second inlet of pump chamber
222a first outlet of pump chamber
222b second outlet of pump chamber
224 gear wheel
224b gear section of gear wheel
224c locking protrusion of gear wheel
225 pump chamber
226a first lobe of pump chamber
226b second lobe of pump chamber
227a first inner wall portion
227b second inner wall portion
227c notch
228a first suction volume
228b second suction volume
229a first expelling volume
229b second expelling volume
230 pump rotor
231 apex
231a apex line/apex sealing line of pump rotor
232 convex curved line/convex curved sealing line of pump rotor
232a convex curved surface
234a sliding/guiding surface on pump rotor/rigid rotor core of pump rotor
234b gear section on pump rotor/rigid rotor core of pump rotor
235 drive pinion
235a eccentric sliding disk of drive pinion
235b drive coupling of drive pinion
236 (rigid) rotor core of pump rotor
236a undercut geometry of rotor core
236b recesses as access for over-mold to undercut geometry
237 (elastic or elastomeric) over-mold of pump rotor
237a (pressured) gas reservoir/blister within or covered by over-mold
238 internal sealing lip
300 giving set
310 first fluid conduit
311 first end of first fluid conduit
312 second end of first fluid conduit
320 upstream section of first fluid conduit
330 (flexible) downstream section of first fluid conduit
335 (flexible) helical winding of first fluid conduit
340 second fluid conduit
341 first end of second fluid conduit
342 second end of second fluid conduit
350 Luer-Lock (at (first) end of first fluid conduit)
360 connector at (second) end of second fluid conduit
370 fluid reservoir
375 pre-filled fluid reservoir
380 tag on (pre-filled) fluid reservoir
400 patient
α (alpha) rotational difference between within which the first inlet is already closed and the first outlet is not yet opened

1. A trochoidal medicament pump device comprising:
a pump body;
an in-port for entering a medicament to be administered;
an out-port for administering a medicament;
a pump rotor having a triangular convex shape and having an eccentric trajectory upon rotation within the pump body;
wherein the pump rotor has three equidistant apexes each two thereof are connected with a convex curved line;
wherein the pump body has a pump chamber;
wherein the pump chamber has a first lobe forming a first inner wall portion of a pump volume of the pump chamber with a first inlet in fluid communication with the in-port and a first outlet in fluid communication with the out-port with the pump rotor eccentrically rotating so that the rotor with the first wall portion forms upon rotation a first suction volume in fluid communication to the first inlet and a first expelling volume in fluid communication to the first outlet,
wherein the pump rotor has a rigid rotor core and an elastomeric over-mold over at least sealing lines along the apexes and sealing lines along the convex curved lines,
wherein the over-mold is made of a biocompatible elastomeric material of a first material class,
wherein the first inner wall portion is made of a biocompatible material of a second material class,
wherein the outer dimensions of the pump rotor with the over-mold are larger than the corresponding inner dimensions of contacted portions of the pump chamber,
wherein the apexes of the eccentrically rotating pump rotor trace a trochoidal line corresponding to a trochoidal shape that is equivalent or larger than the corresponding inner dimensions of the pump chamber.

2. The trochoidal medicament pump device of claim 1, wherein the rigid rotor core has an undercut geometry wherein the undercut geometry along each a convex curved line has an undercut orthogonal to the rotational axis of the rotor and wherein in particular the rigid rotor core along each a convex curved line has a plurality of recesses being adapted for providing access for an over-mold to the undercut geometry.

3. (canceled)

4. The trochoidal medicament pump device of claim 1, wherein the first material class is a group consisting of liquid silicone rubber LSR, ethylene propylene diene monomer rubber EPDM, thermoplastic elastomer TPE, thermoplastic polyurethane PU butyl rubbers and nitrile rubbers.

5. The trochoidal medicament pump device of claim 1, wherein the first material class is a self-lubricating liquid silicone rubber LSR.

6. The trochoidal medicament pump device of claim 1, wherein the first material is a liquid silicone rubber LSR which is treated with a medical grade and bio compatible lubricant, in particular a medical grade and bio compatible silicone oil.

7. The trochoidal medicament pump device of claim 1, wherein the over-mold is made of liquid silicone rubber LSR with a hardness between 10 and 100 Shore (ShA), in particular between 20 and 80 Shore (ShA), in particular between 40 and 60 Shore (ShA), in particular of 50+/−5 Shore (ShA).

8. The trochoidal medicament pump device of claim 1, wherein the rotor has an internal sealing lip which is provided on a at least one side of the rotor which is orthogonal to the rotational axis of the rotor, wherein the internal sealing lip is provided on the respective side radially between a gear section of the rotor and the convex curved lines.

9. The trochoidal medicament pump device of claim 1, wherein the second material class is a group consisting of polyethylene terephthalate PET, polycarbonate PC, Tritan, high density polyethylene HDPE, COC (cycloolefin copolymer), PBT, PPS, and PEI.

10. The trochoidal medicament pump device of claim 1, wherein the biocompatible material of the second material class is a Tritan compounded with a medical grade absorber and wherein in particular the absorbent is adapted for absorbing an optical radiation of a welding laser, in particular an optical radiation of a wavelength between 960 nm and 1000 nm, in particular a wavelength of 980 nm+/−10 nm.

11. (canceled)

12. The trochoidal medicament pump device of claim 1, wherein the rotor core is made of a material of the second material class group filled with reinforcing fibers and wherein in particular the rotor core is made of a material including PBT and 30 weight % glass fibers.

13. (canceled)

14. The trochoidal medicament pump device of claim 1, wherein the outer dimensions of the pump rotor with the over-mold are larger than the corresponding inner dimensions of contacted portions of the pump chamber by a factor, which factor is between 1.01 and 1.1, in particular between 1.01 and 1.05, in particular between 1.02 and 1.03, wherein in particular the factor in the radial direction is between 1.01 and 1.1, in particular between 1.01 and 1.05, in particular between 1.02 and

1. 03, and wherein in particular the factor in the axial direction is between 1.01 and 1.1, in particular between 1.01 and 1.05, in particular between 1.01 and 1.03.

15. (canceled)

16. (canceled)

17. The trochoidal medicament pump device of claim 1, wherein over-mold at each of the convex curved lines has a closed gas reservoir, wherein the gas reservoir is filled with gas under pressure higher than atmospheric pressure, in particular at a pressure between 1.2*10\hat{ }5 Pa and 2.0*10\hat{ }5 Pa, in particular at a pressure of 1.3*10\hat{ }5 Pa+/−0.2*10\hat{ }5 Pa and wherein in particular the gas reservoir has a volume between 1 mm\hat{ }3 and 20 mm\hat{ }3, in particular between 1 mm\hat{ }3 and 10 mm\hat{ }3, in particular 2 mm\hat{ }3+/−0.5 mm\hat{ }3.

18. (canceled)

19. The trochoidal medicament pump device of claim 1, wherein the first inlet and the first outlet end at the first inner wall portion of the pump volume of the pump chamber so that upon eccentrically rotating the pump rotor at least one of the apexes in rotation direction lies between the first inlet and the first outlet and that a rotational difference between a rotational position of the pump rotor where an apex has just crossed the first inlet and a rotational position of the pump rotor where another apex just starts crossing the first outlet is not more than 14°, in particular not more than 10°, in particular between 6° and 10°.

20. The trochoidal medicament pump device of claim 1, wherein the pump chamber has a trochoidal shape with said first lobe and a second lobe forming a second inner wall portion of a pump volume of the pump chamber with the pump rotor eccentrically rotating with the apexes traveling along a trochoidal line corresponding to the trochoidal shape, thus forming upon rotation a second suction volume and a second expelling volume wherein in particular the pump body at at least one axal side has a cover covering and sealing the pump chamber, wherein the cover in an area over the second lobe-has a recess which is recessed in axial direction compared to an area over the first lobe, so that the outer dimensions of the pump rotor with the over-mold are larger than the corresponding inner dimensions of contacted portions of the pump chamber by a factor, and the factor in the area of the second lobe is smaller than the factor in the area of the first lobe, wherein in particular the recess recessed in axial direction in the area over the second lobe is between 0.01 mm and 0.2 mm, in particular between 0.05 mm and 0.1 mm, in particular 0.1 mm+/−0.03 mm, wherein in particular an interface between the pump chamber and the cover provides a unique mounting position of the cover onto the pump chamber, wherein in particular the interface is formed by a keyhole protrusion at the cover which engages a keyhole at a side of the pump chamber, such that the cover can be placed over the pump chamber in only one position, and wherein in particular the cover is provided with an axially protruding rim protruding from the side of the cover facing the pump chamber.

21-25. (canceled)

26. The trochoidal medicament pump device of claim 20, wherein the second inner wall portion has in a circumferential direction at least section-wise a notch formed therein forming upon rotation of the pump rotor a pressure compensation bypass between the second expelling volume and the second suction volume.

27. The trochoidal medicament pump device of claim 20 wherein the pump chamber in the pump volume of the second lobe has a second inlet in fluid communication with the in-port and a second outlet in fluid communication with the out-port with the pump rotor eccentrically rotating so that at least three distinct portions, of the rotor are in permanent sealing contact with the first inner wall portion of the first lobe and second inner wall portion of the second lobe thus forming upon rotation a second suction volume in fluid communication to the second inlet and a second expelling volume in fluid communication to the second outlet, wherein in particular the second inlet is in fluid communication with the in-port and the second outlet is in fluid communication with the out-port.

28. A giving set for providing a medicament from a reservoir to a patient the giving set comprises:
a trochoidal medicament pump according to claim 1;
a first fluid conduit being with one end fixedly connected to the out-port and with the other end fixedly connected to a connector, in particular to at least one of a Luer-lock and an ENFIT.

29. The giving set of claim 28, wherein the first fluid conduit has an upstream section and a flexible downstream section, wherein the flexible downstream section is more flexible than the upstream section, wherein in particular the first fluid conduit is made of a flexible tube of substantially constant diameter and wall thickness, and the flexible downstream section is formed by a pre-formed helical winding of the flexible tube, the geometry thereof providing higher flexibility than a non-helical portion.

30. The giving set of claim 28, wherein the giving set further comprises a second fluid conduit being with one end fixedly connected to the in-port and with the other end fixedly connected to a connector being adapted for being releasable connected to a fluid reservoir or with the other end fixedly connected to a pre-filled fluid reservoir, wherein in particular the prefilled reservoir is gas volume free prefilled with a liquid.

31. (canceled)

32. The giving set of claim 28, wherein the giving set further comprises a tag representative for the liquid in the pre-filled reservoir and being adapted to be read out by a tag reader of a medicament pump driving device.
